(19) United States
(12) Patent Application Publication
(10) Pub. No.: US 2023/0263809 A1
(43) Pub. Date: Aug. 24, 2023

(54) FOOD SUPPLEMENT FOR ALZHEIMER

(71) Applicant: FOLIUM BIOSCIENCES EUROPE B.V., WEESP (NL)

(72) Inventors: Daniela Oana TRAMBITAS, WEESP (NL); Geert Feye WOERLEE, WEESP (NL)

(73) Assignee: FOLIUM BIOSCIENCES EUROPE B.V., WEESP (NL)

(21) Appl. No.: 17/786,924

(22) PCT Filed: Dec. 2, 2020
(86) PCT No.: PCT/EP2020/084339

§ 371 Date: Jun. 17, 2022

(30) Foreign Application Priority Data
Dec. 24, 2019 (EP) 19219673.1
Sep. 15, 2020 (EP) 20196317.0

(51) Int. Cl. A61K 31/00; A61K 31/575; A23L 33/00; A61P 25/28

(52) U.S. Cl. CPC A61K 31/658; A61K 31/575; A23L 33/40; A61P 25/28

(57) ABSTRACT

The present invention is in the field of a Food supplement for Alzheimer. Alzheimer's disease (AD), and likewise associated or similar discomforts, is a chronic neurodegenerative disease that usually starts slowly and gradually worsens over time. The present invention provides a food supplement for preventing Alzheimer's disease, for delaying Alzheimer's disease, for decline of ageing, or combinations thereof.

### RELATED APPLICATIONS

This application is a U.S. national entry of PCT Patent Application No. PCT/EP2020/084339 having international filing date of Dec. 2, 2020, which claims the benefit of priority of European Patent Applications No. 20 196 317.0, filed Sep. 15, 2020, and No. 19 219 673.1, filed Dec. 24, 2019. The entire contents of the above-referenced applications and of all priority documents referenced in the Application Data Sheet filed herewith are hereby incorporated by reference for all purposes.

### FIELD OF THE INVENTION

The present invention is in the field of a Food supplement for Alzheimer. Alzheimer's disease (AD), and likewise associated or similar discomforts, is a chronic neurodegenerative disease that usually starts slowly and gradually worsens over time. The present invention provides a food supplement for preventing Alzheimer's disease, for delaying Alzheimer's disease, for decline of ageing, or combinations thereof.

### BACKGROUND OF THE INVENTION

Deficits in learning and memory problems are generally associated with ageing. A most consistent change during healthy ageing in human's brain structure is shrinkage in brain volume and expansion of the ventricular system. There is substantial evidence suggesting that the activity of the brain endocannabinoid system (ECS) declines with ageing, which is considered due to CB1 receptor expression and coupling to G proteins being reduced in the brain tissue of mammals which are getting older.

In some case, this decline is even promoted by additional neurodegenerative disorders, such as Alzheimer (AD). The AD may be signalled by cognitive impairment in early life stages. This disease is characterized by a progressive neurological disorder which is considered to be provoked mainly by the deposition of Amyloid-β deposits in the senile plaques, neurofibrillary tangles, neuroinflammation which leads to loss of brain volume and a progressive decline in brain functions, particularly memory. A key role in the development of the disease is attributed to the disturbance of the cholesterol-transport in the brain, which lead progressively to the AD.

Thus, a better understanding of such processes may lead to prevention of the molecular and cellular degradation processes, offering novel routes for therapy and/or prevention.

Recent publications have reported clinical researches which evidenced that exposure to chronic low dosage of Δ⁹-tetrahydrocannabidiol (THC) may reverse practically the age-related decline in cognitive performances. THC has been found also to promote the removal of the toxic clumps of Amyloid-β deposits in the brain. The memory seems to be improved when brain cholesterol-transport is activated or re-activated and AD effects are evidently diminished. However, the THS is found to be partly active only.

It is considered that alternatives to this exposure or improvements thereof may be needed.

Some examples of recent publications are given below. WO 2019/159176 A1 recites a composition comprising as the only active agents a combination consisting of a cannabinoid and eucalyptol, e.g., such a fixed-dose combination, which is useful for treatment of a neurological or a movement disorder such as Parkinson's disease, Alzheimer's disease, or ALS, or an injury to the nervous system; and uses thereof. WO 2019/186355 A1 recites a composition comprising at least one cannabinoid receptor type-2 selective agonist, at least one substance having antioxidant properties, and at least one tissue function modulator. Said composition has proved to effectively treat diseases characterised by high oxidative stress, inflammatory state, and tissue dysfunction. WO 99/32097 A2 recites a method of preventing and/or delaying the onset of Alzheimer's disease in an animal comprises administering to the animal a phytosterol composition. A composition useful for preventing and/or delaying the onset of Alzheimer's disease in an animal comprises a phytosterol composition. KR 2018 0130268 A recites a pharmaceutical composition for preventing, ameliorating or treating neurodegenerative diseases, and more specifically, relates to the pharmaceutical composition and a health functional food for preventing, ameliorating or treating the neurodegenerative diseases comprising sargassum horneri extract as an active ingredient. In order to achieve an object of the present invention, the present invention provides the pharmaceutical composition for preventing, ameliorating or treating neurodegenerative degeneracy comprising the sargassum horneri extract as the active ingredient. CN 107 488 509 A recites a method for extracting the essential oil from the flower of Elaeagnus angustifolia by using a supercritical CO2 fluid extraction method, and belongs to the field of deep processing of natural products. A purpose of the invention is to provide a method for extracting the essential oil from the flower of Elaeagnus angustifolia by using supercritical CO2. The method comprises: picking the flower of Elaeagnus angustifolia, drying, feeding into an extraction tank, and extracting, wherein the extraction temperature is 46 DEG C, the extraction time is 240 min, and the extraction pressure is 26 MPa. According to the present invention, the method has advantages of fast extraction, high extraction rate and prolonged storage period, and is widely used in the fields of cosmetics and food.

The present invention therefore relates to a food supplement, a dosage comprising said food supplement, and a medicament, which solve one or more of the above problems and drawbacks of the prior art, providing reliable results, without jeopardizing functionality and advantages.

### SUMMARY OF THE INVENTION

The present invention relates to a food supplement for Alzheimer wherein the supplement comprises 0.1-90 wt. % of at least one active cannabinoid, preferably 0.5-60 wt. %, more preferably 1-35 wt. %, even more preferably 2-20 wt. %, such as 5-10 wt. %, wherein the cannabinoid is of phyto, endo, or synthetic origin, 0.1-90 wt. % of at least one active phytosterol, preferably 0.5-60 wt. %, more preferably 1-35 wt. %, even more preferably 2-20 wt. %, such as 5-10 wt. %, wherein the phytosterol is extracted from biological species, such as from algae, or a pharmaceutically acceptable salt, or a pharmaceutically acceptable derivative thereof, or a tautomer thereof, or a solvate thereof, and likewise to a food supplement per se, e.g. for non-medical use. Typically no other active ingredients are present.

It has been found that one way to activate the brain cholesterol-transport is to make use of phytosterols, which can take over the process, and can be up-taken easily by the human body. In this way one avoids the negative impact of some synthetic activators, which can induce sever health side effects. Phytosterols considered are the Saringosterols and fucosterols, which may be found in specific macro algae, typically of the order Fucales, and preferably of the family Sargassaceae, such as Sargassum Seaweed, and of the order Laminariales, specifically of the family Lassoniaceae, such as Ecklonia species, such as Ecklonia cava, and Ecklonia stolonifera. The ratio Saringosterols/Fucosterols may be between 0.01 to 0.8, depending on a type of the seaweed used for extraction. These seaweeds are however not very tasty and have a strong odour. The uptake of this Saringosterols and fucosterols, and especially 24(S)-Saringosterol, and (3S,8S,9S,10R,13R,145,17R)-10,13-Dimethyl-17-[E,2R)-5-propan-2-ylhept-5-en-2-yl]-2,3,4,7,8,9,11,12,14,15,16,17-dodecahydro-1H- cyclopenta[a]phenanthren-3-ol, is found to diminish the Amyloid-β depositions up to 70%, and is found to help restore neurological behaviours without registering any side effects caused normally, such as by the synthetic LXRα/β activators. Inventors have found a clear benefit of having a diet rich in phytosterols, such as obtained from Seaweed, combined with a controlled low dosage of Δ⁹-tetrahydrocannabidiol (THC). It is an advantage that relatively low dosages can be used. The above combination may be considered, however in practice it is found to be rather difficult to accomplish, as a minimum amount of seaweed which should be part of a daily dietary ration should be around ten grams. Apart from the fact that not all seaweed types contain this preferred saringosterol, such as Sargassumsterol, the availability of these seaweeds may also be rather rare, whereas a concentration of the phytosterol in the seaweed may also be limited.

Distribution of the cannabinoids is also very limited and well controlled in a lot of countries. It is noted that their activity can be easily reduced, such as by utilizing a wrong extraction procedure, or improper purification. A food supplement containing both active ingredients (in high purity) in specific ratios could be a welcome approach to be utilized by elderly persons or for those diagnosed with AD disease, such as in early stages of the disease.

Both claimed active components have biological origin and inventors have found that these can be easily extracted by using supercritical CO₂. It has been found that high purity compounds are obtained, with virtually no impurities, such as <1 wt. %, in active form, and of good quality. The pressurized gas process is found to deliver natural extracts free of chemical solvents, and can therefore be used directly as food supplements, such as in combination with edible oils or fats, or by encapsulation utilizing edible bio-degradable capsules originated in natural polymers like polysaccharides or waxes, fats, or mixtures thereof. Also, it is found that natural actives extracted from algae together have a synergic positive effect when utilized as such without any post fractionation. The loading of the actives in the capsule material can be between 0.1 and 60% and the ratio between the cannabidiols and the phytosterols can also vary between 0.1 to 100%.

The active cannabinoid is of phyto origin, endo origin, or synthetic origin. It is extracted from biological species, typically the Cannabaceae family, preferably the genus Cannabis, such as Cannabis sativa, Cannabis indica, Cannabis ruderalis. The phytosterol is extracted from biological species, such as for Fucales, preferably from brown algae such as the Sargassaceae family, such as from the Sargassum genus.

In a second aspect the present invention relates to a method of providing a food supplement according to the invention, comprising a critical CO₂ extraction method. In the method pre-dried milled seaweed is used, which is contacted with CO₂ near supercritical conditions. The extraction takes place under controlled temperature and pressure. The seaweed extract may contain a mixture of Saringosterols and Fucosterols in approximate ratios of 0.01 to 0.8 Saringosterol/Fucosterol. When using this extract, the daily intake, depending on the Saringosterol concentration in the pure extract, can be reduced to only 10-35 mg extract/day. This extract complies with a daily recommended concentration of 600-700 μg Saringosterol, so about 2-6 wt. % saringosterol. On top of this the extract is also rich in Fucosterol.

In an exemplary embodiment of the present method the algae may be selected from macro algae, typically of the order Fucales, such as of the family Fucaceae, such as of Fucos Vesiculosis, of the family Phaeophyceae, such as Hijiki, of the family Ascophyllum, such as A. nodosum and preferably of the family Sargassaceae, such as Sargassum Seaweed, and of the order Laminariales, specifically of the family Lassoniaceae, such as Ecklonia species, such as Ecklonia cava, and Ecklonia stolonifera.

In a third aspect the present invention relates to a dosage comprising a food supplement according to the invention, comprising 0.1-60 wt. % food supplement, and 1-1000 mg of as active ingredient, wherein the active ingredient comprises at least one active cannabinoid, wherein the cannabinoid is of phyto, endo, or synthetic origin, and at least one active phytosterol, wherein the phytosterol is extracted from biological species, preferably 2-500 mg active ingredient, more preferably 5-200 mg active ingredient, such as 10-100 mg active ingredient. A recommended dose now is approximately 2-3.5 g/day of dry seaweed (Sargassum) or 600-700 micrograms/day pure extract in combination with cannabinoids

In a fourth aspect the present invention relates to a food supplement according to the invention, for use as a medicament for preventing Alzheimer's disease, for delaying Alzheimer's disease, for decline of ageing, for restoring brain tissue, for treatment of a person with a genetically increased chance for Alzheimer's disease, for treating a degenerative disease, such as atherosclerosis, for lowering a blood cholesterol level, for anticancer, for depletion of cancer tumor, for reducing blood platelet aggregation, for antioxidative effect, for anti-diabetic effect, for anti-obesity, or combinations thereof.

Thereby the present invention provides a solution to one or more of the above-mentioned problems and drawbacks.

Advantages of the present description are detailed throughout the description.

Use of the verb “to comprise” and its conjugations does not exclude the presence of elements or steps other than those stated in a claim. The article “a” or “an” preceding an element does not exclude the presence of a plurality of such elements. The mere fact that certain measures are recited in mutually different dependent claims does not indicate that a combination of these measures cannot be used to advantage.

The invention further pertains to a method or process comprising one or more of the characterising features described in the description and/or shown in the attached drawings. The various aspects discussed in this patent can be combined in order to provide additional advantages. Furthermore, some of the features can form the basis for one or more divisional applications.

### DETAILED DESCRIPTION OF THE INVENTION

The present invention relates in an aspect to a food supplement according to claim 1.

In an exemplary embodiment the present food supplement may be for preventing Alzheimer's disease, for delaying Alzheimer's disease, for decline of ageing, or combinations thereof.

In an exemplary embodiment of the present food supplement the cannabinoid may be selected from Tetrahydrocannabinolic acid THCA, Cannabidiolic acid CBDA, Cannabigerolic Acid CBGA, cannabichromenic acid CBCA, cannabinol acid (CBNA), tetrahydrocannabivarin acid (THCVA), cannabielsoin acid (CBEA), cannabicycol acid (CBLA), cannabicitran acid (CBTA), there iso-forms, their cyclic forms, derivatives thereof, and their active neutral forms, such as Δ⁹-tetrahydrocannabidiol (THC). The term “iso-form” is considered to relate to a commonly used terminology in chemistry, depicting a possible naturally occurring transformation of a chemical substance with the same chemical formula of one form thereof into another, such as structural isomers and stereoisomers. A similar transformation may occur to cyclic forms and straight forms, typically under removal/addition of a water molecule.

In an exemplary embodiment of the present food supplement the phytosterol may be selected from saringosterols, preferably Sargassum Seaweed saringosterol, especially 24(S)-saringosterol.

In an exemplary embodiment of the present food supplement the at least one cannabinoid and phytosterols are each individually >90% pure, preferably >99% pure.

In an exemplary embodiment of the present food supplement a total amount of active cannabinoid and active phytosterol may be between 0.1-60 wt. %, preferably between 0.5-40 wt. %, more preferably between 1-30 wt. %, such as between 2-15 wt. %.

In an exemplary embodiment of the present food supplement the at least one cannabinoid and phytosterols may each individually be extracted by super-critical CO₂.

In an exemplary embodiment of the present food supplement the food supplement may comprise <5 ppm solvent, preferably <2 ppm solvent, such as <1 ppm solvent.

In an exemplary embodiment the present food supplement may comprise 5-50 wt. % of edible oil, and edible fat, preferably 10-25 wt. %.

In an exemplary embodiment the present food supplement may be provided in a capsule, where the capsule material is selected from polysaccharides, waxes, fats, proteins, and mixture thereof. The capsule is found to improve a release profile and may provide a taste masking effect in addition.

In an exemplary embodiment of the present food supplement a ratio between weights of cannabinoid and phytosterol may be from 1:1000 to 1000:1, preferably from 1:200 to 200:1, more preferably from 1:100 to 100:1, even more preferably from 1:30 to 30:1, such as from 1:10 to 10:1.

In an exemplary embodiment the present food supplement may further comprise at least one carrier selected from oils, powders, and mixtures thereof. Plant material like Seaweed can be also considered to be encapsulated as it is.

In an exemplary embodiment of the present food supplement may further comprise one or more of a pharmaceutically acceptable excipient, such as selected from fillers, binders, and disintegrants. A disintegrant is any material that may be added to the present food supplement to make it disintegrate, and thereby releasing the active ingredient, typically on contact with water.

The one or more of the above examples and embodiments may be combined, falling within the scope of the invention.

### EXAMPLES

The below relates to examples, which are not limiting in nature, showing the benefits of the invention.

| Running | | Solvent | | 24R,S- | | 24R,S- | |
|---|---|---|---|---|---|---|---|
| time | Sample | mixtured | Fuco | Saringo | Fuco | Sariongo | |
| [min] | [mg] | [ml] | [μg] | [μg] | [μg/mg] | μg/mg | Saringo/Fuco |
| 60 | 900 | 10 | 73 | 17.021 | 81.442 | 18.912 | 0.2322 |
| 120 | 1100 | 10 | 78 | 16.620 | 70.650 | 15.109 | 0.213857471 |
| 180 | 800 | 10 | 60 | 13.850 | 75.310 | 17.313 | 0.229881741 |
| 240 | 700 | 10 | 38 | 8.890 | 53.749 | 12.700 | 0.236281671 |

10 ml of CCl₃/MeOH was used each time. 10 μl of worked-up material was obtained.

The invention is further detailed by the accompanying figures, which are exemplary and explanatory of nature and are not limiting the scope of the invention. To the person skilled in the art it may be clear that many variants, being obvious or not, may be conceivable falling within the scope of protection, defined by the present claims.

### FIGURES

The invention although described in detailed explanatory context may be best understood in conjunction with the accompanying figures.

FIG. 1a-b show a Saringosterol and a fucosterol.

FIG. 2 shows Focus veziculosus.

FIG. 3 shows an experimental set-up.

FIG. 4 shows milled seaweed.

FIG. 5a,b Seaweed extract collected from separator.

FIGS. 6a-c show Extract and CBD, Coating phase, and Emulsion.

FIG. 7 shows dry powder.

FIG. 8a,b show SEM micrographs (different magnifications of the coated particles.

FIG. 9: drying process.

FIGS. 10a,b obtained products.

### DETAILED DESCRIPTION OF THE FIGURES

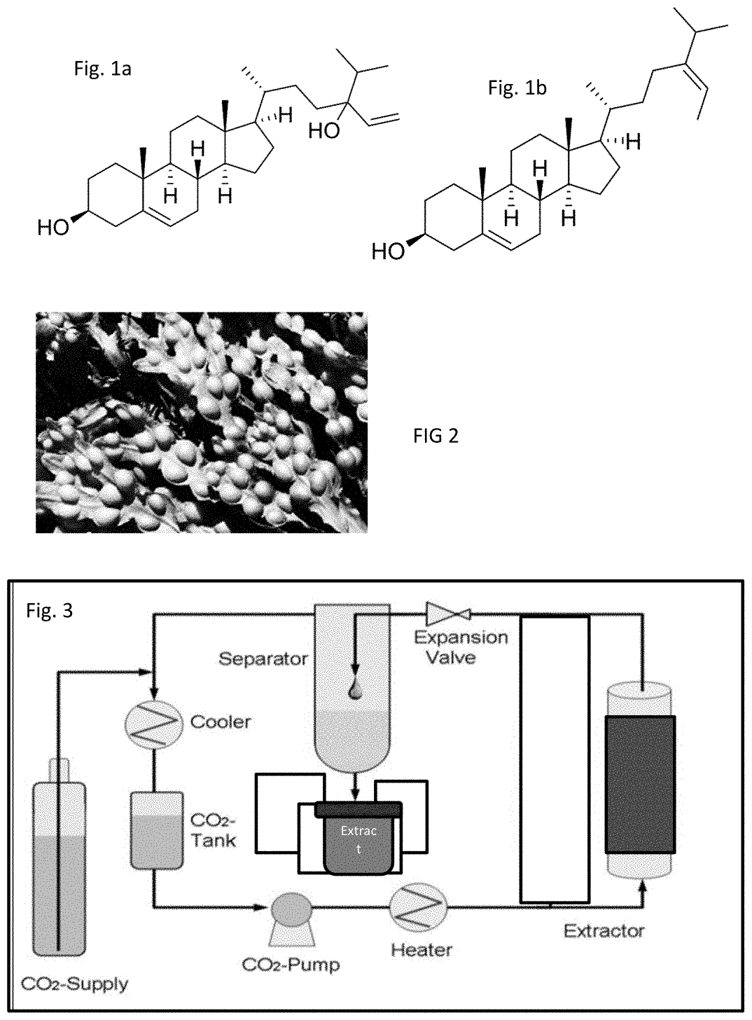

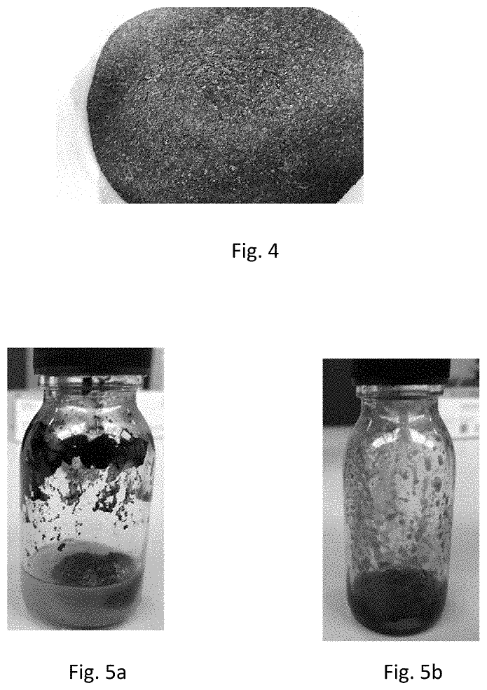

FIG. 1a-b show a Saringosterol (PubChem CID 14161394) and a fucosterol (CAS 17605-67-3).

Seaweed Extraction Tests

Example 1

Tested Material: Bladder wrack known also as rockweed or bladder fucus (Focus veziculosus) harvested form North Sea (see FIG. 2).

It is currently used as a dietary supplement as an accelerator for basal metabolism or as a mean to muscle recovery from intensive physical training. It also helps weight loss and its control.

Due to its rich extracts like fucoidans, alginic acids, and laminarin has wide recommendations for its positive results in reducing blood glucose levels, or reducing the plasma cholesterol levels. It has a high content of iodine which give a certain restriction for usage specially for people with thyroid disorders, hypertension, bleeding problems or pregnant women. However, the iodine content recommends this seaweed also for hypothyroidism.

Milled Seaweed: 400 g

Super critical (Sc) CO₂ extraction setup was used for extracting the active ingredients from this seaweed (see FIG. 3). 400 g of milled dry algae is placed into a 1L extractor and CO2 under supercritical conditions is brought in contact with the algae. The active ingredients from the algae are solubilized into the scCO₂ at operating conditions (30000 kPa (300 bar), 40° C., for 4 h). And the CO₂ flow rich in extract is passed into the separator where because of solubility differences (due to pressure differences) the extracts are separated from the scCO₂ and further collected in the extract vessel (FIG. 5a,b).

The extract contains Fucosterol and Saringosterol (rations S/F=0.23). The extract contains also some water which is co-extracted together with the sterol fraction.

The water fraction is removed and the sterol fraction is mixed with CBD supplied via separate extraction form hemp flowers (following the present procedure) a coating material containing modified starch and maltodextrin (Capsule:MD5:MD20). The ration used in this example was: approx. 87% coating comprise of approximately 35% MD20, 8.7% MDS, and 43.5% Capsule. The loading used was approximately 13%, comprising of 10.4% seaweed extract (with a ration Saringosterol/Fucosterol of 0.23) and 2.5% CBD (99% purity) extracted also by scCO₂ from dry hemp flowers.

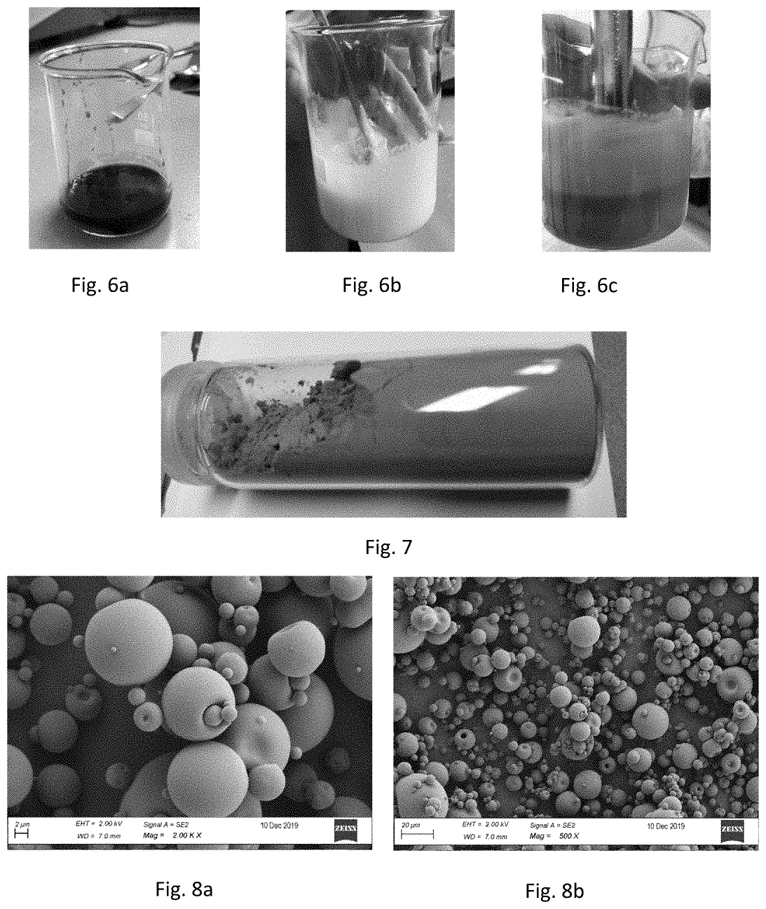

The two phases are mixed to form emulsion. FIGS. 6a-c show Extract and CBD, Coating phase, and Emulsion. The mixture coating/extract forms a stable emulsion which is further used to dry it by scCO₂ spraying process.

Dry powder (FIG. 7) consists of encapsulated extracts and CBD in a coated matrix of Modified starch (Capsule) and maltodextrin (MD20 and MD5).

During the drying process, based on the material crystallization properties, the coating matrix will form around the extract mixture a solid thin protective layer which entrap the sterols and CBD inside the capsule material.

SEM micrographs (FIGS. 8a,b) show different magnifications of the coated particles.

The encapsulation efficiency can also be computed based on PL/PT determinations (Journal of AOAC International, 2005, Vol 88, pg 1271-1274). PL/PT stands for active ingredient found on the exterior of the particles and total active ingredients fund in a specific amount of encapsulated powder.

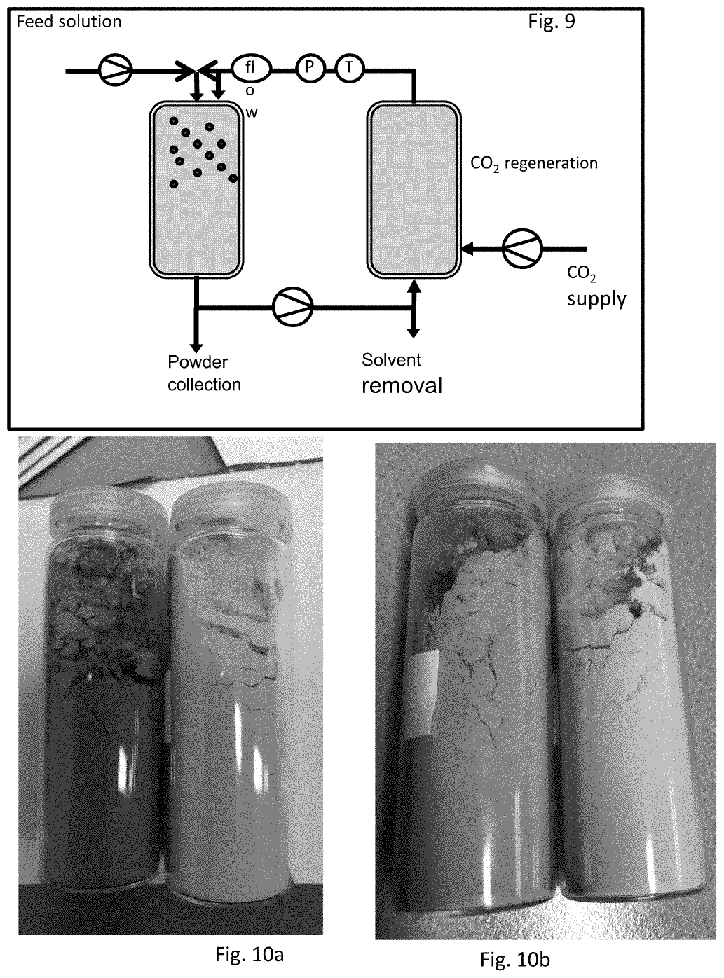

The spray drying process is done in a closed system with CO₂ continuous recirculation (FIG. 9), and Schematic presentation of the encapsulation process.

Example 2

Same extraction process as in example 1.

For loading we used only seaweed extract without CBD.

The product is lighter in colour compared to the one produced with CBD. FIGS. 10a,b show obtained products.

Example 3

Sargasum recovery from Fucus Vesiculosis using scCO2 and Hemp oil.

The use of ethanol as modifier in sccO2 extraction almost double the Saringosterol extraction from Fucus Vesiculosis (from 0.242 μg/mg to 0.427 μg/mg). Other oils like MCT (from coconut) or hemp oil (cold pressed) proved to have a positive role in scCO2 extraction of Saringosterol from Fucus Vesiculosis seaweed as well. In the case of hemp oil our experiments proved that the high concentration of Fucosterol in the extract is a result of Fucosterol presence in the hemp oil itself.

| | Co-solvent | | | |
|---|---|---|---|---|
| | used in | | | |
| Seaweed type | scCO2 | Fucosterol | Iso-Fucosterol | Saringosterol |
| (Name) | extraction | μg/mg | μg/mg | μg/mg |
| Asc Nodosum | — | 58.77 | 0.597 | 0.084 |
| Hijiki | | 126.37 | 0.928 | 5.571 |
| Hijiki | C2H5OH | 114.09 | 0.815 | 7.272 |
| Brown algae | | 30.31 | 0.377 | 0.124 |
| Fucus | | | | |
| Fucos | | 55.67 | 0.662 | 0.239 |
| Vesiculosis | | | | |
| Fucos | C2H5OH | 48.57 | 0.697 | 0.383 |
| Vesiculosis | | | | |

| | Co-solvent | Fucosterol | Iso-Fucosterol | Saringosterol |
|---|---|---|---|---|
| | used | μg/μl | μg/μl | μg/μl |
| Fucos | MCT | 95.62 | | 1.479 |
| Vesiculosis | (coconut oil) | | | |
| Fucos | Hemp oil | 354.43 | | 1.480 |
| Vesiculosis | | | | |
| Fucos | Hemp oil | 321.68 | 29.12 | 1.107 |
| Vesiculosis | | | | |
| — | Hemp oil | 242.89 | 30.76 | — |

This result in the following conclusions: the combination between hemp oil and algae extract is beneficial. The results showed that in the hemp oil is sufficient Fucosterol. Literature mention Fucosterol in clinical phase 2 studies. So far, clinical studies demonstrated that dietary intake of plant sterols might help to lower blood cholesterol levels. Fucosterol also shows a cholesterol lowering effect by competing with cholesterol absorption, which is the same effect as plant sterols. In addition, fucosterol shows anti-cancer, antioxidative, and anti-diabetic effects. Fucosterol is present in brown algae in relatively large quantities: 0.9 mg/g to 13.4 mg/g dry weight. Now we have proved that other algae can yield important fucosetrol amounts next to Saringosterol. More over other plants like Hemp can be also an important source of Fucosterol. (Anti-obesity and anti-diabetic activities of algae. Fucosterol is also partially co-extracted from seaweed as proved in the current patent. Thus in combination with algae extract, Fucosterol may bring additional benefits in curing Alzheimer disease as well as depletion of some cancer tumours.

It is found that the sterols present in hemp oil are steroid aliphatic alcohols which proved to act positively in lowering the cholesterol as well as reducing the platelet aggregation. Phytol and tocopherol both have not only remarkable antioxidant activities but also beneficial results against degenerative diseases such as atherosclerosis and Alzheimer's. Next to that hemp oil has additional nutritional benefits due to high level of vitamin (A,C,E), beta-carotene, minerals (like phosphorus, K, Ca, Mg) and its excellent balance of polyunsaturated fatty acids. None of prior art specifically relates to Fucosterol presence in the hemp oil. In this example inventors have shown its presence in the seaweed extract which aims to be used to reduce the effects of Alzheimer's disease.

The extract is used further as such or encapsulated (see previous example).

1. A food supplement for use in the treatment of Alzheimer, the supplement comprising
0.1-90 wt. % of at least one active cannabinoid, wherein the origin of the cannabinoid is selected from phyto, endo, and synthetic origin, wherein the cannabinoid is extracted from biological plant species,
0.1-90 wt. % of at least one active phytosterol, wherein the phytosterol is extracted from biological species,
wherein the food supplement is selected from a food supplement as such, from a pharmaceutically acceptable salt, and from a solvate thereof,
wherein all wt. % are based on a total weight of the food supplement.

2. The food supplement for use in the treatment of Alzheimer according to claim 1, wherein the food supplement is for at least one of preventing Alzheimer's disease, delaying Alzheimer's disease, and maintaining activity of the brain endocannabinoid system (ECS).

3. The food supplement for use in the treatment of Alzheimer according to claim 1, wherein the cannabinoid is selected from Tetrahydrocannabinolic acid (THCA), Cannabidiolic acid (CBDA), Cannabigerolic Acid (CBGA), cannabichromenic acid (CBCA), cannabinol acid (CBNA), tetrahydrocannabivarin acid (THCVA), cannabielsoin acid (CBEA), cannabicycol acid (CBLA), cannabicitran acid (CBTA), there iso-forms, their cyclic forms, and their active neutral forms.

4. The food supplement for use in the treatment of Alzheimer according to claim 1, wherein the phytosterol is selected from saringosterols.

5. The food supplement for use in the treatment of Alzheimer according to claim 1, wherein the at least one cannabinoid and phytosterols are each individually >90% pure, and wherein a total amount of active cannabinoid and active phytosterol is between 0.1-60 wt. %.

6. The food supplement for use in the treatment of Alzheimer according to claim 1, wherein the at least one cannabinoid and phytosterols are each individually extracted by super-critical CO₂.

7. The food supplement for use in the treatment of Alzheimer according to claim 1, wherein the food supplement comprises <5 ppm solvent.

8. The food supplement for use in the treatment of Alzheimer according to claim 1, comprising 5-50 wt. % of edible oil, and edible fat.

9. The food supplement for use in the treatment of Alzheimer according to claim 1, in a capsule, where the capsule material is selected from polysaccharides, waxes, fats, proteins, and mixture thereof.

10. The food supplement for use in the treatment of Alzheimer according to claim 1, wherein a ratio between weights of cannabinoid and phytosterol is from 1:1000 to 1000:1.

11. The food supplement for use in the treatment of Alzheimer according to claim 1, further comprising at least one carrier selected from oils, powders, and mixtures thereof.

12. The food supplement for use in the treatment of Alzheimer according to claim 1, further comprising one or more of a pharmaceutically acceptable excipient.

13. A food supplement comprising
0.1-90 wt. % of at least one active cannabinoid, wherein the origin of the cannabinoid is selected from phyto, endo, and synthetic origin, wherein the cannabinoid is extracted from biological plant species,
0.1-90 wt. % of at least one active phytosterol, wherein the phytosterol is extracted from biological species, wherein the food supplement is selected from a food supplement as such, from a pharmaceutically acceptable salt, and from a solvate thereof,
wherein all wt. % are based on a total weight of the food supplement.

14. A method of providing a food supplement, the food supplement comprising 0.1-90 wt. % of at least one active cannabinoid, wherein the origin of the cannabinoid is selected from phyto, endo, and synthetic origin, wherein the cannabinoid is extracted from biological plant species, 0.1-90 wt. % of at least one active phytosterol, wherein the phytosterol is extracted from biological species, wherein the food supplement is selected from a food supplement as such, from a pharmaceutically acceptable salt, and from a solvate thereof, wherein all wt. % are based on a total weight of the food supplement, the method comprising super critical CO₂ extraction under increased pressure of >10 kPa, a temperature of >20° C., during a period of >1 h.

15. The method according to claim 14, wherein the phytosterol is extracted from algae, wherein the algae is selected from macro algae of the order Fucales, of the family Phaeophyceae, of the family Ascophyllum, and of the family Sargassaceae, and of the order Laminariales.

16. A dosage comprising a food supplement, the food supplement comprising 0.1-90 wt. % of at least one active cannabinoid, wherein the origin of the cannabinoid is selected from phyto, endo, and synthetic origin, wherein the cannabinoid is extracted from biological plant species, 0.1-90 wt. % of at least one active phytosterol, wherein the phytosterol is extracted from biological species, wherein the food supplement is selected from a food supplement as such, from a pharmaceutically acceptable salt, and from a solvate thereof, wherein all wt. % are based on a total weight of the food supplement,
the dosage comprising 0.1-60 wt. % food supplement, and 1-1000 mg of as active ingredient, wherein the active ingredient comprises the at least one active cannabinoid.

17. The food supplement according to claim 1 in a method selected from at least one of the treatment of Alzheimer from preventing Alzheimer's disease, from delaying Alzheimer's disease, from maintaining activity of the brain endocannabinoid system (ECS), from restoring CB1 receptor expression and coupling to G proteins, from treatment of a person with a genetically increased chance for Alzheimer's disease, from treating a degenerative disease, from lowering a blood cholesterol level, from anti-cancer treatment, from depletion of cancer tumour, from reducing blood platelet aggregation, from providing anti-oxidative effect, from providing anti-diabetic effect, and from providing anti-obesity.
